(19) United States
(12) Patent Application Publication
(10) Pub. No.: US 2007/0053056 A1
(43) Pub. Date: Mar. 8, 2007

(54) Viewing device with orientating imaging device

(76) Inventor: Charles Nicolas Granville Charlesworth, Newcastle-Upon-Tyne (GB)

Correspondence Address: NIXON PEABODY LLP - PATENT GROUP, CLINTON SQUARE, P.O. BOX 31051, ROCHESTER, NY, 14603-1051, US

(21) Appl. No.: 10/575,840

(22) PCT Filed: Oct. 14, 2004
(86) PCT No.: PCT/GB04/04365

§ 371 Date: Sep. 1, 2006

(30) Foreign Application Priority Data
Oct. 14, 2003 (GB) 0324108.0

(51) Int. Cl. A61B 1/00

(52) U.S. Cl. 359/367

(57) ABSTRACT

The invention relates to the provision of an optical device having first and second viewing passages which are selectively spaced apart to suit the interocular distance between a user's eyes. The device also included at least one image capturing device to allow a digital image to be captured. The invention provides a means whereby adjustment between the first and second viewing passages can be taken into account when capturing the image so as to reduce the problems caused by the angular adjustment.

The invention to which this application relates is apparatus and a method for incorporating at least one imaging device, or more than one, within a set of binoculars in order to allow the capturing of data for an image representative of the view to which the binoculars are directed. The imaging device, or other devices connected thereto, can be used for subsequent display and/or storage of said image or images for further processing.

In some iterations of this invention the said imaging device may be an electronic sensor or a photochemical film. As is well known, binoculars comprise first and second image “passages”, a first passage provided with a front lens passing to an eye piece for the first of the user's left or right eyes to which the same is placed and the second passage positioned with an eyepiece for placement at the other of the user's eyes. Typically, each of the passages has an objective or zoom lens hereinafter referred to as an objective lens and focusing means to allow the view to be focussed.

In some arrangements one or more mirrors or prisms may be positioned within each passage between the objective lens and eyepieces. Intermediate, and connecting the first and second passages is a frame with adjustment means which allows pivotal movement of the first and second passages about a pivotal axis which is typically common to both passages and which runs parallel with the longitudinal axes of the first and second passages. The movement about the pivotal axis allows the viewing passages to be moved with respect to each other such that the eyepieces are moved to a position in front of the user's eyes. Said movement typically results in the spacing between the eyepieces being increased or decreased to permit the user to obtain optimum viewing of the selected object in accordance with the spacing of the user's eyes.

It is also possible to view the object with an electronic view screen if the incident light passing through the objective lens is directed onto a suitable sensor and the output processed into a digital format in an appropriate manner. The sensor may be fixed to the casing of one or both passages but it should be appreciated that it is not a necessity of this invention that the image captured by a sensor or sensors is displayed on any view screen within or attached to the binocular casing.

While the angle of orientation of the first and second viewing passages is required to be adjusted for the user's physical viewing requirements, the orientation, and possibility to change the same, causes a significant problem in attempting to capture the image viewed through each of the viewing passages as, if the image sensor or sensors are mounted in a fixed position on their respective passages, the sensor position moves with the movement of the passages.

The conventional solution to overcome this problem is to mount the sensor or sensors in a manner that it is fixed relative to the hinge at the pivotal axis for the viewing passages of the binocular casing and/or use a lens or lenses other than those of the objective lenses of each of the above described passages. An inherent problem associated with this solution is that of trying to provide for the relative alignment of the viewed image using the two passages and the image captured by the sensor or sensors using their own independent lens or lenses. The person skilled in the art of the use of binoculars will appreciate that the greater the magnification offered then the greater the requirement for accurate alignment.

An additional problem with this solution is that of focusing. Adjustment for focus is typically required to be made in normal use and the greater the magnification provided by the binoculars, the greater the need for said adjustment. Unless the conventional focusing operation of the binocular product is linked to a focusing system of the camera or sensor for the camera, it is inevitable that the image quality will not be of best quality.

With suitable positioning of the sensor within a passage and any accompanying lens systems the use of the conventional focusing mechanism of the binoculars will also keep in focus the image falling onto the sensor thus overcoming the limitations of a camera with its own independent lens system. A preferred arrangement is to mount the sensor within the above mentioned passage or passages. It should be noted that if a sensor is mounted in a fixed position within the passage and the passages adjusted relative to each other during the normal process of adjusting the interocular spacing of the eyepieces then the orientation of the sensor or sensors will change. No matter what the original alignment of the sensor to the axis of the binoculars, any subsequent adjustment for interocular spacing will result in an effective rotation of the captured image relative to the axis of the binoculars which is typically the horizontal plane. It should be noted that if one sensor is fitted to each passage then the images will be rotated in opposite directions.

This image rotation is a problem for single image capture but it is a particular problem if one of the aims is to use the captured images from the first and second passages as an image pair to form a 3 Dimensional image pair as it is important, for this use to be successful, that the two images used have substantially the same orientation such as with respect typically to a common horizontal plane. If this is not achieved the 3D image cannot be successfully created without extensive post capture processing and adjustment.

The aim of the invention is to provide a method and apparatus to allow the interocular spacing of a set of binoculars to be adjusted while at the same time ensuring that the adjustment does not adversely affect the capturing of the viewed images electronically or on photochemical film. Said provision is made by providing a mechanism or mechanisms that result in the images remaining substantially in a predefined plane with which the apparatus is designed to operate no matter how much adjustment for interocular spacing is made.

In a first aspect of the invention there is provided a viewing device including first and second viewing passages, each passage having a main body and an eyepiece assembly at one end thereof, said passages and/or eyepiece assemblies selectively movable to allow positional adjustment to suit the interocular distance of the user and characterised in that said viewing device includes at least one imaging device to allow the selective capture and/or storage of data representing the images viewable through the first and/or second passages.

In one embodiment of the invention there is provided a viewing device including first and second viewing passages, each passage having a main body and an eyepiece assembly at one end thereof, said eyepiece assemblies selectively movable relative to their main body to allow positional adjustment to suit the interocular distance of the user, at least one viewing passage including at least one imaging device to allow the selective capture and/or storage of data representing the images viewable through the first and/or second passages and characterised in that the eyepiece assemblies are adjustable independently of their respective main bodies of the first and second viewing passages and the at least one imaging device.

Typically the main bodies of the first and second viewing passages are provided as a single unit.

Typically the imaging device remains in a fixed position with respect to the main bodies of the first and second viewing passages. This allows the imaging device to capture data representative of the view through the passages at a position in front of the eyepiece assembly in each passage thereby ensuring that the orientation of the imaging device or devices remains constant regardless of the adjustments made to the eyepiece assemblies and so the orientation of the images which it or they are capturing remain in the same orientation regardless of the orientation adjustments.

By providing the eyepiece assemblies to be respectively adjustable with respect to the main bodies or body of the viewing passages so the necessary interocular adjustments can be made to suit particular user requirements.

Typically the light path in each viewing passage passes from the front lens to the eyepiece via a series of relatively angularly disposed mirrors or prisms with some of said mirrors or prisms mounted in the fixed part of each passage and others mounted in the relatively movable eyepiece part.

In a further aspect of the invention there is provided a viewing device including first and second viewing passages, each viewing passage having an eyepiece assembly at one end thereof, said first and second viewing passages adjustable with respect to one another to allow the interocular spacing between the eyepiece assemblies to be adjusted to suit user requirements, said viewing device including at least one imaging device for capturing the images from the first and/or second viewing passages and characterised in that the device includes a detection means for detecting the relative angles of orientation of the first and second viewing passages, and control means for said at least one imaging device which receives an electronic signal indicative of the orientation which is detected.

In one embodiment the electronic control means for each imaging device receives the signal and then, as the imaging device is known to have moved in conjunction with the viewing passage, electronically adjusts the orientation of the image or images which are captured by the imaging device to ensure it remains in substantial alignment with a horizontal or other predetermined plane which may or may not be adjusted by the user.

In a further embodiment the signal indicative of the orientation of the viewing passages is utilised by control means for the imaging device or devices to orientate the imaging devices such that the imaging device or devices, rather than following the movement of the viewing passages, are in fact counter moved to effectively maintain the same in the required orientation with regard to a datum which may, for example, be a common horizontal plane. In this arrangement the control means include the actuation of a gearing arrangement with respect to which the imaging devices can be mounted and hence moved. In another arrangement the adjustment may be effected by a simple gearing system with no electronic signals or control.

In whichever embodiment, the binoculars can include a “reset” or adjustment function which ensures that, if selected, typically by the user, the first and second viewing passages, and imaging means return to a “default” or other setting selected by the user thereby ensuring that any “drift” in the respective angular position is removed.

Typically a number of viewing configurations can be adopted to allow the capture of the data from the viewing passages by the imaging device or devices. The particular configuration chosen can be dependant upon any or any combination of the cost restrictions on the product, the particular orientation compensation apparatus used and/or the required clarity of view.

In one embodiment, one of the viewing passages will become blank to the eye looking through that passage while the image data for the view through that passage is captured as the object light falls onto the imaging device.

Typically the imaging device is a sensor.

In this embodiment either one of the mirrors or prisms used to define the light path is movable to divert the light path between a path to the sensor and a path to the eyepiece. In one embodiment the mirror or prism can be positioned to move out of the light path permitting the light to fall directly onto the sensor, possibly via a lens and/or supplementary mirrors or prisms.

Typically, in whichever embodiment, the capturing of the image data by the imaging device is activated by at least one actuation of a trigger by the user, such that the user, when they are looking at a view which they wish to capture, can actuate the trigger means to commence the image data capture by further actuation of the trigger or other device. The image data capture can be in respect of still images or video (moving) images.

In one embodiment the trigger actuation involves a two stage process, the first stage upon a first actuation is to move the mirror or prism and the second trigger actuation initiates the image data capture. Typically the mirror or prism moves back after the trigger is released.

In the embodiments where two imaging devices are provided, one for each viewing passage, then in one configuration one or two electronic view screens are provided showing the output of one of the imaging devices at any given time or, if two screens are provided, showing the output from the respective imaging devices or alternatively just one of the imaging devices.

In an alternative configuration, as the light path moves along the viewing passage a beam splitter is located in the viewing passage such that the light path is split into two components, a first component which is directed to the imaging device and a second component which is directed to the eyepiece.

In a further aspect of the invention there is provided a viewing device in the form of a set of binoculars including first and second viewing passages connected by two or more pivoting joints characterised in that movement of the passages for interocular spacing adjustment results in no change to the orientation of the imaging devices.

Typical of such arrangement would be a parallelogram-type configuration.

In a yet further aspect of the invention, there is provided a viewing device including first and second viewing passages, each viewing passage or component thereof selectively movable around a pivot axis to allow positional adjustment to suit the interocular distance of the user, and including at least one imaging device to allow the selective capture and/or storage of data representing the images viewable through the first and/or second passages, characterised in that said at least one imaging device is positioned to receive light in an axis perpendicular to said pivot axis.

In this arrangement, the images captured by the imaging device are correctly orientated irrespective of the angle between the viewing passages, such that electronic correction of the images, or movement of the imaging device is not required.

Specific embodiments of the invention are now described with reference to the accompanying drawings wherein

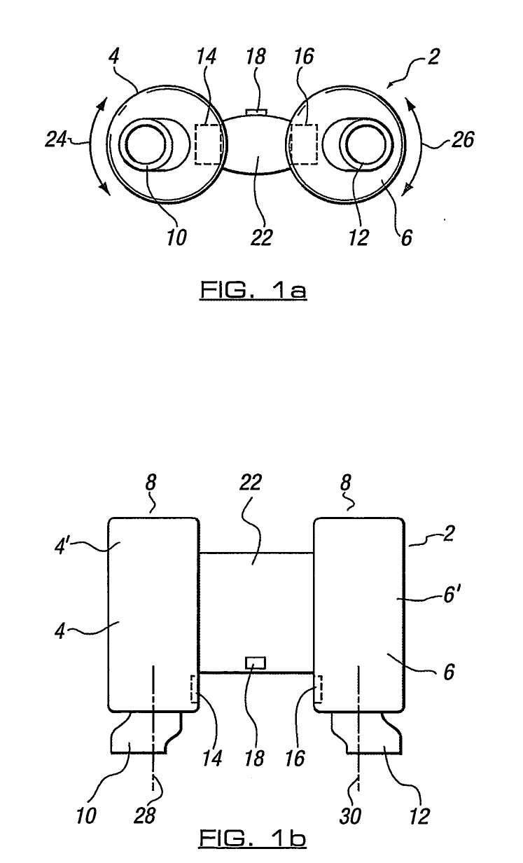

FIGS 1a and b illustrate a first embodiment of the invention where the eyepiece assemblies are relatively movable;

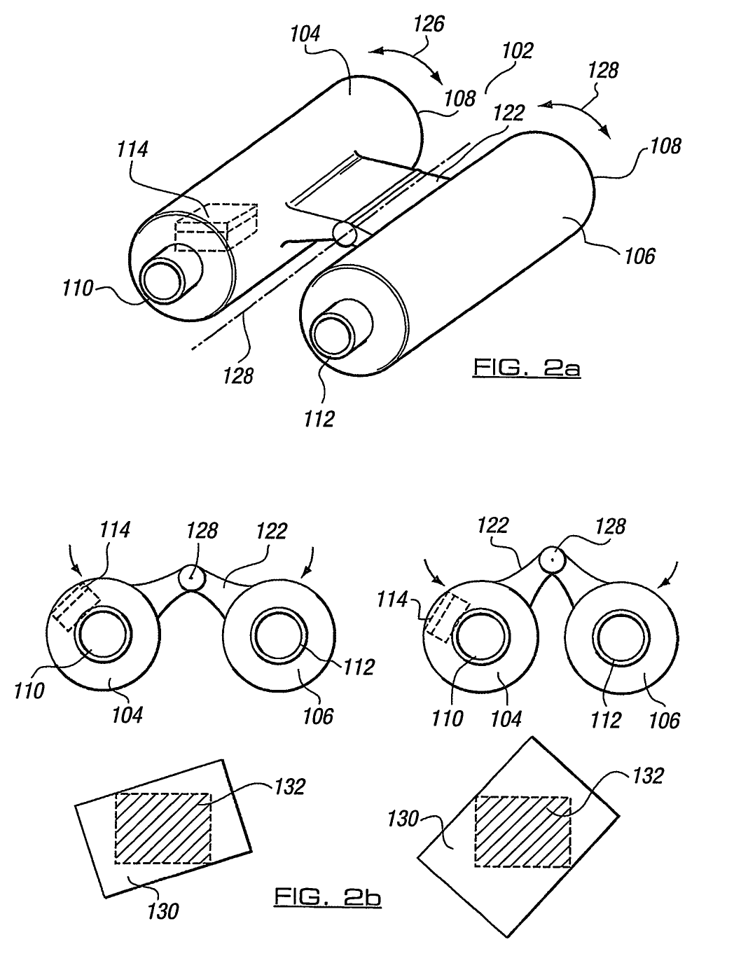

FIGS. 2a and b illustrates a further embodiment of the invention; and

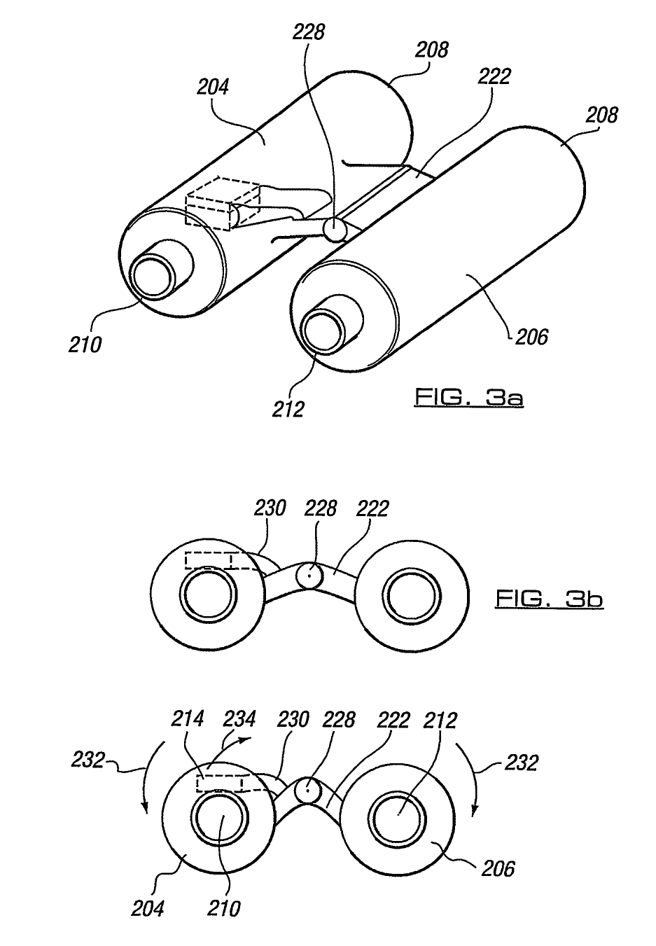

FIGS. 3a and b illustrate a yet further embodiment of the invention.

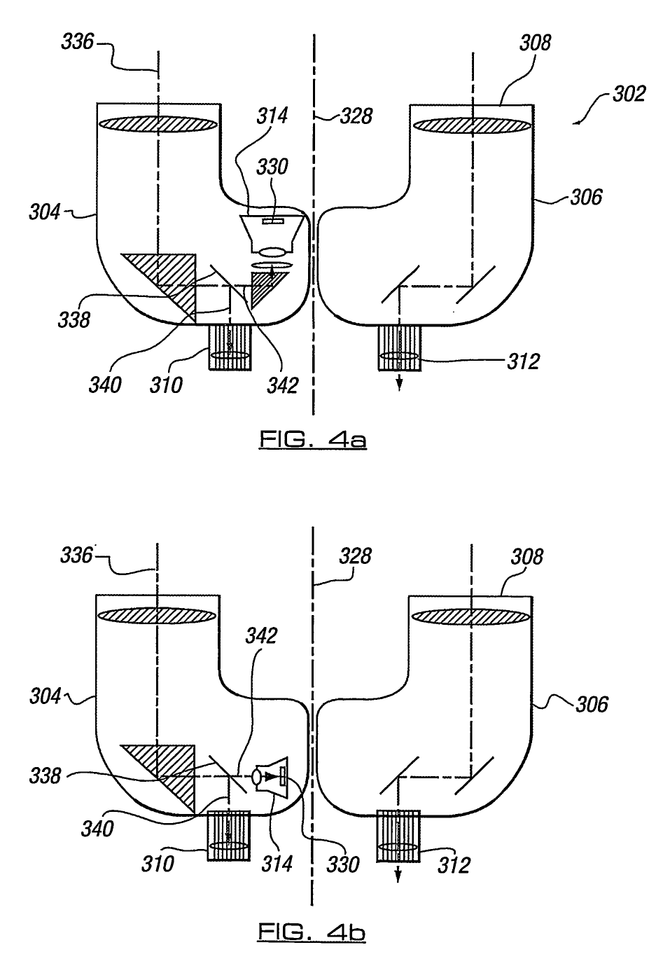

FIGS. 4a and b illustrate schematic views of embodiments of the invention.

Referring firstly to FIGS. 1a and b there is illustrated a first embodiment of the invention with the views showing the set of binoculars 2 in plan and end elevation from the direction of the user when using the same. The binoculars include first and second viewing passages 4,6 each having a front end 8 through which light enters the passage and passes along the same to the eyepiece assemblies 10, 12 for the viewing passages 4,6 respectively. Each eyepiece assembly has a lens which can be placed to the persons eyes and focussing means. The light path from the front 8 of each passage allows the view at which the binoculars are pointed to be carried to the respective eyepiece assembly 10, 12.

Each viewing passage has at least one magnifying lens positioned therein to allow magnification of the view and the light path is controlled and directed along the viewing passage by a mirror and/or prism configuration. The exact configuration of the mirrors and prisms can be selected to suit particular requirements and is in itself not of significance to the invention as herein described. Furthermore the mirror configuration can be replaced or partially replaced by an electronic viewing system to suit particular requirements. The binoculars also include, in this embodiment, imaging devices 14, 16 in the form of a sensor provided on each, which allow the capture of data representative of the view carried by the light path along the viewing passage or passages. Each imaging device acts as a camera in that it allows the selective capture of data to allow the generation of a still image or alternatively the capturing of data over a period of time to generate a moving video image. In whichever embodiment the capture of the data is achieved by the user actuation of a trigger means, in this case button 18 which causes each sensor to be activated and exposed to the light path in the respective viewing passage. The captured data can then be stored in a memory for subsequent processing and/or display via a display screen (not shown in this embodiment). In this embodiment the imaging device 14 is provided to capture data from the viewing passage 4 by the movement of a mirror in the viewing passage to temporarily divert the light path onto the same, and imaging device 16 captures data from the viewing passage 6 by the movement of a mirror in that passage to temporarily divert the light path on to that imaging device.

However, as has already been explained, alternative configurations can be provided to allow the imaging devices to be exposed to the light paths when necessary for the capturing of data and it should be appreciated that each of the configurations previously described can be interchangeable with the embodiments described in these figures.

A problem which is common to all of these embodiments is how to take into account the changes in position of the eyepiece assemblies and conventionally the viewing passages which can occur when the user adjusts the same with respect to a pivotal axis normally provided in the frame 22 which joins the viewing passages 4,6 together. The adjustment is required to be undertaken to move the eyepieces as shown by the arrows 24,26 to allow the same to be positioned suitably for placement in front of the eyes of the user. This adjustment would normally lead to the orientation of the imaging devices also changing which is problematic for the reasons already set out.

In FIGS. 1a and b the problem is overcome by allowing the main bodies 4′,6′ of the viewing passages 4,6 to be fixed with respect to each other and thereby, in conjunction with the frame 22 form a single unit. The eye piece assemblies 10,12 are each independently movable about respective axes 28,30 as shown to allow the position of the same to be adjusted to match the users eye position. As the imaging devices 14,16 are mounted on the main body portions, which do not move, so the relative orientation of the same is fixed throughout and the problem is removed. As already stated, a suitable mirror and/or prism arrangement is provided which allows the eyepiece assemblies to be adjusted while at the same time ensuring that in each of the positions to which the eyepiece assembly can be adjusted the light paths can still be directed along the viewing passages to the eyepiece assemblies.

Turning now to FIGS. 2a and b there is provided a further embodiment of the invention. FIG. 2a illustrates the set of binoculars 102 which again comprise viewing passages 104, 106 with respective front ends 108 and eyepiece assemblies 110,112. In this case the eyepiece assemblies are provided as a fixed part of the binoculars with the respective viewing passages joined by the frame 122. In this embodiment the frame 122 includes a pivot axis 128 and the viewing passages can be moved about this pivot axis as shown by arrows 124, 126 to position the viewing passages and hence the eyepiece assemblies to suit the users eye spacing. In this example only one imaging device 114 is provided to allow the selective capture of the image from the light path along the viewing passage 104.

In the position shown the imaging device 114 is provided in the required horizontal plane and therefore the data captured via the imaging device will be in the correct orientation for display of the image. However as shown in FIG. 2b, if the viewing passages are moved about the pivot axis 128 then the imaging device 114 will also change orientation such that the same will no longer be in the required horizontal plane. The binoculars are therefore provided with angle detection means which detect the relative angle and position of the viewing passages at each instance. As a result of the angle detection, the processing of the captured data can be tailored to suit as is now described.

In each of the examples shown in FIG. 2b there is shown the area of image 130 which is captured by the imaging device and the orientation of the same with regard to the position of the imaging device in the viewing passage as shown. In accordance with this embodiment, the angle of orientation of the imaging device is detected and taken into account when selecting the data which lies within an area 132 at the correct orientation and which is a subset of the area 130. Thus, although the data which is used is smaller in area, the data is in the correct orientation and so the data for area 132 is used to generate the display of the image and at the correct orientation.

Referring now to FIGS. 3a and b there is illustrated a further embodiment of the invention. In this embodiment the same components are provided as shown in FIG. 3a with viewing passages 204, 206, having front ends 208, and eyepiece assemblies 210, 212 respectively. A frame 222 is provided with a pivot axis 228 about which the viewing passages 204, 206 can be rotated selectively by the user.

An angle position detection device is again provided to detect the angle of the viewing passage 204, or if required both passages when two imaging devices are provided. In this case the embodiment is described with respect to one imaging device but can be used with two imaging devices if required.

In this case the imaging device is supported independently of the viewing passage with the support for the same movable such that the imaging device can be moved independently of the viewing passage in which the same is located. The sequence of operation is therefore as follows;

1 Viewing passages positions adjusted about pivot axis 228 to suit user's requirements for eyepiece assembly.

2 Position of the viewing passage(s) detected by angle position detection means

3 Signal generated which is indicative of the angle position and said signal transmitted to control means for the imaging device mount 230

4 The drive means for the mount are activated to move the imaging device into apposition where the same is held in the required orientation, typically in a horizontal plane.

In this manner the imaging device can effectively be moved in the opposite direction to the movement of the viewing passage to ensure that the imaging device remains in the correct orientation as illustrated in FIG. 3b where the binoculars are illustrated in a first position in which the imaging device and viewing passages are in a horizontal plane, and a second position where the viewing passages have been moved as indicated by arrows 232. In turn the imaging device mount is moved by drive means typically a gearing mechanism to move the same in the direction 234 to be held in the required horizontal plane.

In an alternative embodiment the movement of the imaging device can be by a purely mechanical means whereby movement of the viewing passages is translated into reverse movement of the imaging device by a mechanical gearing arrangement. In a yet further arrangement the mount 230 can be mounted on to the pivot axis 228 and therefore be fixed with respect to the same. It will be appreciated that a mechanisms or mechanisms can be provided such that the movement of the imaging device to compensate to the relative movement of the two passages does not alter the light path distance from the objective lens to the imaging device thus ensuring that the image retains its focus. Typical of such arrangement would be a mechanism that rotates as appropriate the imaging device in a plane perpendicular to the light path falling on the imaging device.

Referring to FIG. 4a, there is illustrated a schematic view of an embodiment of the invention as hereinbefore described, illustrating the light path therein. The binoculars 302 include viewing passages 304, 306, having front ends 308, eyepiece assemblies 310, 312 respectively, and an imaging device 314 provided to allow the selective capture of the image from the viewing passage 304. The viewing passages 304, 306 can be rotated selectively by the user about a pivot axis 328. The viewing passage 304 includes a reflective surface or prism 338 to selectively direct incoming light 336 towards the eyepiece assembly 310 as indicated by arrow 340 and/or the imaging device 314 as indicated by arrow 342.

With reference to FIG. 4b, the imaging device 314 is positioned in the viewing passage 304 such that the plane defined by the capture area 330 is parallel to the pivotal axis 328 of the viewing passages 304, 306. The plane of the capture area 330 remains parallel to the axis 328 as the viewing passages 304, 306 are pivoted. Thus the image captured by the imaging device 314 is provided at the correct orientation irrespective of the angle between the viewing passages, and without the requirement for additional correction mechanisms.

It will therefore be appreciated that in each of the embodiments herein described there is provided a solution which allows the data captured by the imaging device from the binoculars to be presented for subsequent viewing in the correct orientation, regardless of the particular position of the eyepieces and/or viewing passages.

1. A viewing device including first and second viewing passages, each passage having a main body and an eyepiece assembly at one end thereof, said passages and/or eyepiece assemblies selectively movable to allow positional adjustment to suit the interocular distance of the user and characterised in that said viewing device includes at least one imaging device to allow the selective capture and/or storage of data representing the images viewable through the first and/or second passages.

2. A viewing device according to claim 1 characterised in that said passages and/or eyepiece assemblies are selectively movable about a pivot axis, and said at least one imaging device is positioned to receive light in an axis perpendicular to said pivot axis.

3. A viewing device according to claim 2 characterised in that said viewing device includes one or more reflective surfaces for guiding light from a viewing passage to an eyepiece assembly and/or said at least one imaging device.

4. A viewing device according to claim 3 characterised in that the reflective surfaces are adjustable to allow user selection of the path of the light between the eyepiece assembly and imaging device.

5. A viewing device according to claim 1 characterised in that the main bodies of the first and second viewing passages are provided as a single unit.

6. A viewing device according to claim 1 characterised in that said at least one imaging device is fixedly positioned with respect to the main bodies of the first and/or second viewing passages.

7. A viewing device according to claim 1 characterised in that said viewing device includes one or more displays to display the captured images.

8. A viewing device according to claim 1 characterised in that said viewing device includes one or more reflective surfaces for guiding light through the main body of each viewing passage to the eyepiece assemblies and/or said at least one imaging device.

9. A viewing device according to claim 8 characterised in that said one or more reflective surfaces are in the form of a series of relatively angularly disposed mirrors, beam splitters and/or prisms.

10. A viewing device according to claim 8 characterised in that said one or more reflective surfaces are mounted in the fixed part of each viewing passage and/or the relatively movable eyepiece part.

11. A viewing device according to claim 8 characterised in that one of the viewing passages will become blank to the eye looking through that passage while the image data for the view through that passage is captured as the object light falls onto the imaging device.

12. A viewing device according to claim 8 characterised in that one or more of said reflective surfaces is movable to guide light to the imaging device and/or the eyepiece assembly.

13. A viewing device according to claim 1 characterised in that the viewing device is provided with actuation means to allow the imaging device to capture images and/or video.

14. A viewing device according to claim 13 characterised in that the actuation means firstly moves any combination of mirrors, prisms and/or lenses from a first position to a second position, and secondly initiates capture.

15. A viewing device according to claim 14 characterised in that the mirrors, prisms and/or lenses move back to the first position when the actuation means is released.

16. A viewing device including first and second viewing passages, each passage having a main body and an eyepiece assembly at one end thereof, said eyepiece assemblies selectively movable relative to their main body to allow positional adjustment to suit the interocular distance of the user, at least one viewing passage including at least one imaging device to allow the selective capture and/or storage of data representing the images viewable through the first and/or second passages and characterised in that the eyepiece assemblies are adjustable independently of their respective main bodies of the first and second viewing passages and the at least one imaging device.

17. A viewing device including first and second viewing passages, each viewing passage having an eyepiece assembly at one end thereof, said first and second viewing passages adjustable with respect to one another to allow the interocular spacing between the eyepiece assemblies to be adjusted to suit user requirements, said viewing device including at least one imaging device for capturing the images from the first and/or second viewing passages and characterised in that the device includes a detection means for detecting the relative angles of orientation of the first and second viewing passages, and control means for said at least one imaging device which receives an electronic signal indicative of the orientation which is detected.

18. A viewing device according to claim 17 characterised in that the control means for said at least one imaging device receives the signal and adjusts the orientation of the images and/or said at least one imaging device.

19. A viewing device according to claim 18 characterised in that the orientation is adjusted to ensure the images and/or said at least one imaging device remain in substantial alignment with a horizontal or other predetermined or user-selectable plane.

20. A viewing device according to claim 19 characterised in that the control means includes gearing arrangements, an electric motor, and/or any combination thereof.

21. A viewing device according to claim 1 characterised in that the viewing device includes a reset or adjustment function to return the first and second viewing passages and/or imaging devices and/or components thereof to a predetermined position.

22. A viewing device in the form of a set of binoculars including first and second viewing passages connected by two or more pivoting joints, and imaging devices mounted in or on the binoculars, characterised in that movement of the passages for interocular spacing adjustment results in no change to the orientation of the imaging devices.

23. A viewing device including first and second viewing passages, each viewing passage or component thereof selectively movable around a pivot axis to allow positional adjustment to suit the interocular distance of the user, and including at least one imaging device to allow the selective capture and/or storage of data representing the images viewable through the first and/or second passages, characterised in that said at least one imaging device is positioned to receive light in an axis perpendicular to said pivot axis.

24. A viewing device according to claim 16 characterised in that the viewing device includes reset or adjustment function to return the first and second viewing passages and/or imaging devices and/or components thereof to a predetermined position.

25. A viewing device according to claim 17 characterised in that the viewing device includes a reset or adjustment function to return the first and second viewing passages and/or imaging devices and/or components thereof to a predetermined position.
